(19) United States
(12) Patent Application Publication
(10) Pub. No.: US 2023/0111766 A1
(43) Pub. Date: Apr. 13, 2023

(54) STRUCTURE INSPECTION METHOD AND STRUCTURE INSPECTION SYSTEM

(71) Applicant: FUJIFILM Corporation, Tokyo (JP)

(72) Inventors: Tadashi KASAMATSU, Ashigarakami-gun (JP); Naoko YOSHIDA, Ashigarakami-gun (JP)

(73) Assignee: FUJIFILM Corporation, Tokyo (JP)

(21) Appl. No.: 18/050,911

(22) Filed: Oct. 28, 2022

Related U.S. Application Data
(63) Continuation of application No. PCT/JP2021/019703, filed on May 25, 2021.

(30) Foreign Application Priority Data
May 29, 2020 (JP) 2020-094328

(51) Int. Cl. G01N 21/95; G01N 29/04; G01N 33/2045; G01N 33/38; G01N 27/82

(52) U.S. Cl. CPC G01N 21/95; G01N 29/045; G01N 33/2045; G01N 33/383; G01N 27/82; G01N 2291/0232; G01N 2291/0289

(57) ABSTRACT

Provided are a structure inspection method and a structure inspection system capable of easily detecting an abnormal location and inspecting an internal state of the abnormal location in detail. The structure inspection method includes: a step of capturing a thermal image of a surface of a structure with an infrared camera; a step of detecting a first region estimated to have an internal abnormality, on the basis of the thermal image; and a step of measuring an internal state of the first region in a case where the first region is detected. In the step of measuring the internal state of the first region, the internal state of the first region is measured by capturing an image that visualizes the internal state of the first region using an electromagnetic wave or an ultrasonic wave.

### CROSS-REFERENCE TO RELATED APPLICATIONS

The present application is a Continuation of PCT International Application No. PCT/JP2021/019703 filed on May 25, 2021 claiming priority under 35 U.S.C § 119(a) to Japanese Patent Application No. 2020-094328 filed on May 29, 2020. Each of the above applications is hereby expressly incorporated by reference, in its entirety, into the present application.

### BACKGROUND OF THE INVENTION

### 1. Field of the Invention

The present invention relates to a structure inspection method and a structure inspection system.

### 2. Description of the Related Art

An infrared photographic method (thermography) is known as a non-destructive inspection method for a structure (for example, JP2005-37366A and JP2016-6398A). In the infrared photographic method, an infrared camera is used to capture a thermal image of a surface of a structure, and existence of an internal abnormality is estimated on the basis of the obtained thermal image. A structure repeats heat absorption from the outside to the inside and heat radiation from the inside to the outside, under an influence of outside air and sunlight. An Abnormal location with a cavity portion functions as a heat insulating layer during heat absorption and heat radiation. As a result, a temperature difference occurs between an abnormal location and a sound location without the abnormality. Therefore, in a case where imaging is performed with the infrared camera, the abnormal location and the sound location are displayed in different colors on the thermal image. Accordingly, it is possible to determine the presence or absence of an internal abnormality occurring in the structure by observing the thermal image. In addition, it is also possible to discriminate an occurrence position of the abnormal location by observing the thermal image because the abnormal location usually occurs locally.

### SUMMARY OF THE INVENTION

However, in the infrared photographic method, while the abnormal location can be easily detected, there is a drawback that an internal state cannot be inspected in detail.

The present invention has been made in view of such circumstances, and an object of the present invention is to provide a structure inspection method and a structure inspection system capable of easily detecting an abnormal location and inspecting an internal state of the abnormal location in detail.

(1) A structure inspection method comprising: a step of capturing a thermal image of a structure with an infrared camera; a step of detecting a first region estimated to have an internal abnormality, on the basis of the thermal image; and a step of measuring an internal state of the first region in a case where the first region is detected.

(2) The structure inspection method of (1), in which the internal state of the first region is measured by capturing an image that visualizes the internal state of the first region, in a case where the first region is detected.

(3) The structure inspection method of (2), in which the internal state of the first region is measured by capturing the image that visualizes the internal state of the first region using an electromagnetic wave or an ultrasonic wave, in a case where the first region is detected.

(4) The structure inspection method of (3), in which the internal state of the first region is measured by capturing the image that visualizes the internal state of the first region using a millimeter wave, a microwave, or a terahertz wave, in a case where the first region is detected.

(5) The structure inspection method of (1), in which the internal state of the first region is measured by a non-contact acoustic exploration method in a case where the first region is detected.

(6) The structure inspection method of any one of (1) to (5), further comprising: a step of measuring the internal state of the first region by hammering the first region, in a case where the first region is detected, in which the internal state of the first region is measured in a case where determination is made that there is no abnormality after the first region is hammered.

(7) The structure inspection method of (6), in which determination is made that there is no abnormality in a case where the first region has no flaking after the first region is hammered.

(8) The structure inspection method of any one of (1) to (7), in which in a case where the structure is a structure made of reinforced concrete, a region estimated to have delamination is detected as the first region on the basis of the thermal image.

(9) The structure inspection method of any one of (1) to (5), further comprising: a step of capturing a visible light image of a surface of the structure with a visible light camera; and a step of detecting a second region estimated to have an internal abnormality, on the basis of the visible light image, in which the internal state of the first region is measured, in a case where the first region and the second region are detected and the first region and the second region are the same region.

(10) The structure inspection method of (9), in which the internal state of the first region is measured by capturing an image that visualizes the internal state of the first region, in a case where the first region is detected.

(11) The structure inspection method of (10), in which the internal state of the first region is measured by capturing the image that visualizes the internal state of the first region using an electromagnetic wave or an ultrasonic wave, in a case where the first region is detected.

(12) The structure inspection method of (11), in which the internal state of the first region is measured by capturing the image that visualizes the internal state of the first region using a millimeter wave, a microwave, or a terahertz wave, in a case where the first region is detected.

(13) The structure inspection method of (9), in which the internal state of the first region is measured by a non-contact acoustic exploration method in a case where the first region is detected.

(14) The structure inspection method of any one of (9) to (13), in which in a case where the structure is a structure made of reinforced concrete, a region estimated to have delamination is detected as the first region on the basis of the thermal image, and a water leakage region is detected as the second region on the basis of the visible light image.

(15) The structure inspection method of (14), further comprising: a step of inspecting soundness of a reinforcing bar in the first region, in a case where the delamination is detected in the first region as a result of measuring the internal state of the first region and a depth of the delamination is within a predetermined range.

(16) The structure inspection method of (15), in which the soundness of the reinforcing bar is non-destructively inspected.

(17) The structure inspection method of (16), in which the soundness of the reinforcing bar is inspected by an electromagnetic induction method.

(18) A structure inspection method comprising: a step of measuring an internal state of an inspection target region for a structure made of reinforced concrete; and a step of inspecting soundness of a reinforcing bar in the inspection target region, in a case where delamination is detected in the inspection target region by the measurement and a depth of the detected delamination is within a predetermined range.

(19) The structure inspection method of (18), in which the soundness of the reinforcing bar is non-destructively inspected.

(20) The structure inspection method of (19), in which the soundness of the reinforcing bar is inspected by an electromagnetic induction method.

(21) A structure inspection system comprising: an infrared camera that captures a thermal image of a structure; and an imaging device that captures an image which visualizes an internal state of the structure, in which in a case where a region estimated to have an internal abnormality in the structure is detected from the thermal image captured by the infrared camera, an internal state of the region is measured by capturing an image that visualizes the internal state of the region with the imaging device.

According to the present invention, an abnormal location can be easily detected, and an internal state of the abnormal location can be inspected in detail.

### BRIEF DESCRIPTION OF THE DRAWINGS

FIG. 1 is a schematic configuration diagram of an inspection system used to inspect a structure.

FIG. 2 is a block diagram showing an example of a hardware configuration of an inspection device body.

FIG. 3 is a block diagram of a function of the inspection device body.

FIG. 4 is a flowchart showing an inspection procedure of a first embodiment.

FIG. 5 is a flowchart showing an inspection procedure of a second embodiment.

FIG. 6 is a flowchart showing an inspection procedure of a third embodiment.

FIG. 7 is a flowchart showing an inspection procedure of a fourth embodiment.

### DESCRIPTION OF THE PREFERRED EMBODIMENTS

Hereinafter, preferred embodiments of the present invention will be described with reference to the accompanying drawings.

### First Embodiment

Here, a case of inspecting the presence or absence of delamination of concrete in a structure made of reinforced concrete, such as a bridge, will be described as an example.

The delamination of concrete refers to a state in which the vicinity of the surface of concrete is delaminated. The delamination of concrete means a state in which the concrete in the vicinity of the surface loses its integrity with the internal concrete due to continuous fissuring occurring inside the concrete or the like.

[System Used to Inspect Structure]

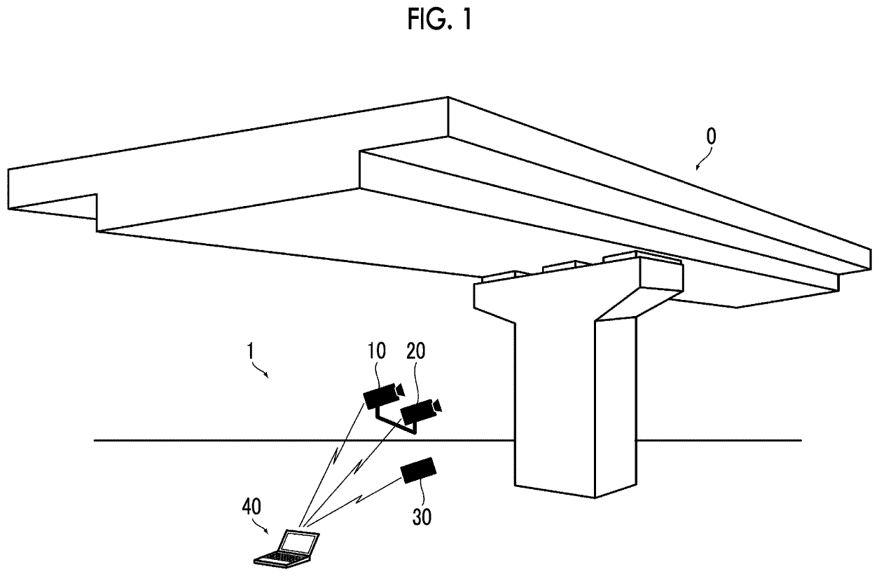

FIG. 1 is a schematic configuration diagram of an inspection system used to inspect a structure.

As shown in FIG. 1, an inspection system 1 of the present embodiment comprises an infrared camera 10, a visible light camera 20, a millimeter wave camera 30, and an inspection device body 40.

The infrared camera 10 captures a thermal image of the surface of a structure O as an inspection target. The thermal image represents the temperature distribution (heat distribution) on the surface of a subject. The infrared camera 10 is communicably connected to the inspection device body 40. The form of communication is not particularly limited. Image data of the thermal image captured by the infrared camera 10 is output to the inspection device body 40.

The visible light camera 20 captures a visible light image of the surface of the structure O as the inspection target. The visible light image is an image obtained by imaging the subject with sensitivity in a visible light wavelength range (generally from 380 nm to 780 nm). As the visible light camera 20, a general digital camera (including a camera mounted on a mobile terminal or the like) on which a complementary metal-oxide semiconductor device image sensor (CMOS image sensor), a charge coupled device image sensor (CCD image sensor) or the like is mounted can be used. In the present embodiment, a digital camera capable of color imaging is used. Therefore, a color image is captured as the visible light image. The color image is an image (a so-called RGB image) having respective intensity values (brightness values) of red (R), green (G), and blue (B) in a pixel unit. The visible light camera 20 is communicably connected to the inspection device body 40. The form of communication is not particularly limited. Image data of the visible light image captured by the visible light camera 20 is output to the inspection device body 40.

The infrared camera 10 and the visible light camera 20 have substantially the same angle of view and image substantially the same range from substantially the same position. For example, the infrared camera 10 and the visible light camera 20 are installed in parallel on the same tripod via a bracket, and image the subject from substantially the same position.

The millimeter wave camera 30 captures a millimeter wave image that visualizes the internal state of the structure O as the inspection target. The millimeter wave camera 30 is one of units that measure the internal state of the structure. Further, the millimeter wave camera 30 is an example of an imaging device that captures an image which visualizes the internal state of the structure. The millimeter wave camera 30 of the present embodiment is composed of a so-called active millimeter wave camera. The active millimeter wave camera irradiates the subject with a millimeter wave, receives the reflected wave, and generates the millimeter wave image that visualizes the internal state of the subject. The millimeter wave is an electromagnetic wave with a wavelength of 1 to 10 mm and a frequency of 30 to 300 GHz. The millimeter wave camera 30, for example, electronically or mechanically scans the subject with a millimeter wave beam to generate a two-dimensional image of the internal state of the subject within the angle of view. A plurality of transmitting antennas and a plurality of receiving antennas are used so that imaging can be speeded up. For example, the two-dimensional image can be generated by arranging the plurality of receiving antennas in one direction and scanning the subject in a direction orthogonal to the arrangement direction. Further, in a case where the plurality of transmitting antennas and the plurality of receiving antennas are used, a so-called multiple input multiple output (MIMO) radar technology can also be adopted. MIMO is a technology that generates more virtual receiving antennas than the number of installed receiving antennas by transmitting signals from the plurality of antennas. The MIMO radar technology is adopted so that the resolution can be further improved. The millimeter wave camera 30 is communicably connected to the inspection device body 40. The form of communication is not particularly limited. Image data of the millimeter wave image captured by the millimeter wave camera 30 is output to the inspection device body 40.

The inspection device body 40 receives the image data output from the infrared camera 10, the visible light camera 20, and the millimeter wave camera 30 to perform processing thereon. The inspection device body 40 includes a computer provided with an operation unit, a display unit, and the like.

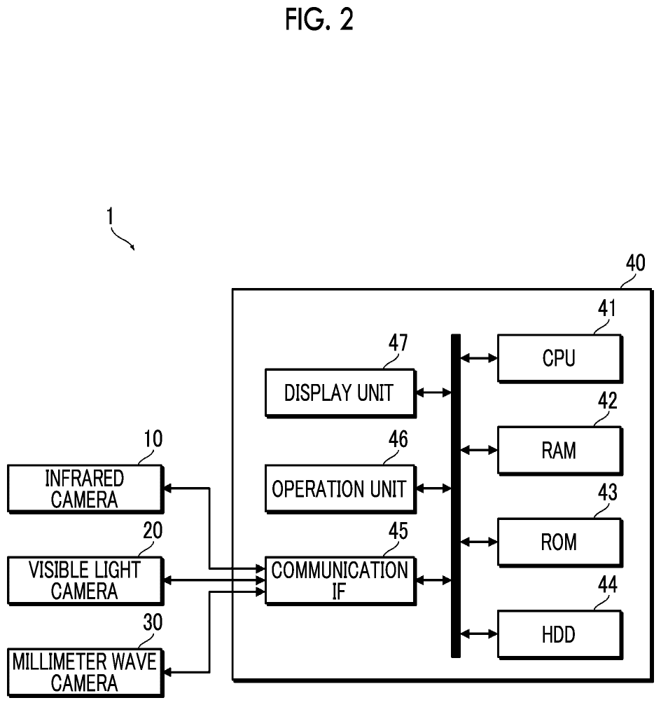

FIG. 2 is a block diagram showing an example of the hardware configuration of the inspection device body.

As shown in FIG. 2, the inspection device body 40 comprises a central processing unit (CPU) 41, a random access memory (RAM) 42, a read only memory (ROM) 43, a hard disk drive (HDD) 44, a communication interface (IF) 45, an operation unit 46, a display unit 47, and the like. Various kinds of data and a program executed by the CPU 41 are stored in the ROM 43 and/or the HDD 44. The operation unit 46 includes, for example, a keyboard, a mouse, and a touch panel. The display unit 47 includes, for example, a liquid crystal display (LCD) and an organic EL display (organic light emitting diode display, OLED display). The infrared camera 10, the visible light camera 20, and the millimeter wave camera 30 are communicably connected to the inspection device body 40 via the communication interface 45.

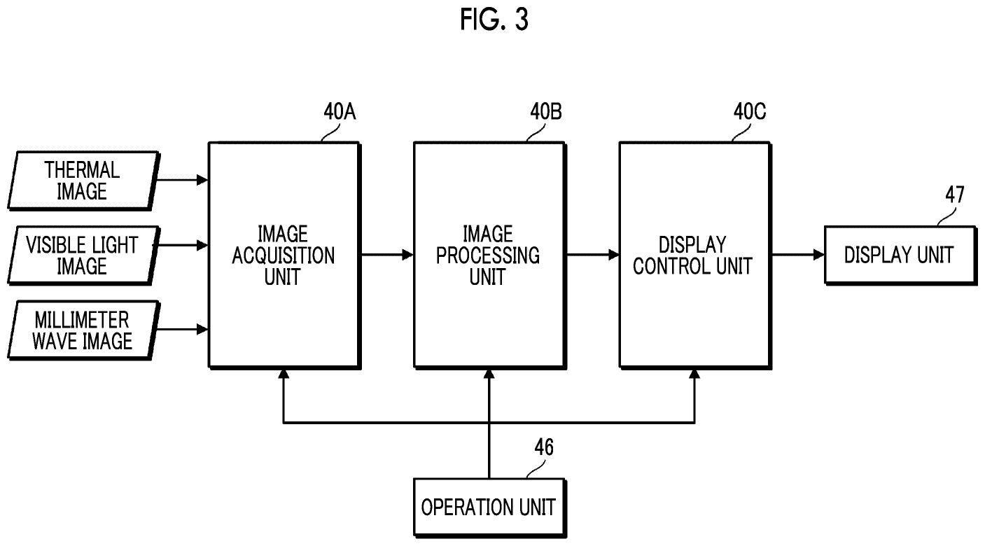

FIG. 3 is a block diagram of the function of the inspection device body.

The inspection device body 40 mainly has functions of an image acquisition unit 40A, an image processing unit 40B, and a display control unit 40C. These functions are realized by the CPU 41 executing a predetermined program.

The image acquisition unit 40A acquires image data obtained by imaging, from each camera, in response to an instruction input from a user via the operation unit 46. Specifically, the image data of the thermal image is acquired from the infrared camera 10, the image data of the visible light image is acquired from the visible light camera 20, and the image data of the millimeter wave image is acquired from the millimeter wave camera 30.

The image processing unit 40B performs predetermined image processing on the image data in response to an instruction input from the user via the operation unit 46. The image processing here includes processing of detecting a region estimated to have delamination from the image, on the thermal image, in addition to processing of generating image data for display.

A well-known technique can be adopted as a technique for detecting the delamination of concrete from the thermal image. For example, a technique for detecting the delamination of concrete from the thermal image using an image recognition model generated through machine learning, deep learning, or the like can be adopted. The type of machine learning algorithm is not particularly limited. For example, an algorithm using neural networks, such as a recurrent neural network (RNN), a convolutional neural network (CNN), and a multilayer perceptron (MLP), can be used. In a case where the image recognition model is used to detect the region estimated to have delamination, the thermal image is preprocessed as necessary. That is, processing (for example, filtering) for improving recognition accuracy is performed.

In a case where processing of automatically detecting delamination from the thermal image has been performed, the image processing unit 40B generates an image including the detection result, as an image for display. For example, an image in which the location of the detected delamination is surrounded by a frame is generated. In addition, the image processing unit 40B generates, as the image for display, an image in which the visible light image and the thermal image are juxtaposed, an image in which the thermal image is superimposed on the visible light image, or the like, as necessary. In a case where the image in which the thermal image is superimposed on the visible light image is generated, for example, an image in which an image as the semi-transparent thermal image is superimposed on the visible light image is generated. Alternatively, an image in which only the region estimated to have delamination is superimposed thereon is generated. In this case, an image in which the region estimated to have delamination is cut out from the thermal image and the cut-out image is superimposed on the corresponding position of the visible light image is generated.

Further, the image processing unit 40B performs processing of correcting parallax generated between the infrared camera 10 and the visible light camera 20, as necessary. The correction of parallax is required, for example, in a case where close-up imaging of the subject is performed.

The display control unit 40C displays the image captured by each camera on the display unit 47, in response to an instruction input from the user via the operation unit 46.

[Structure Inspection Method]

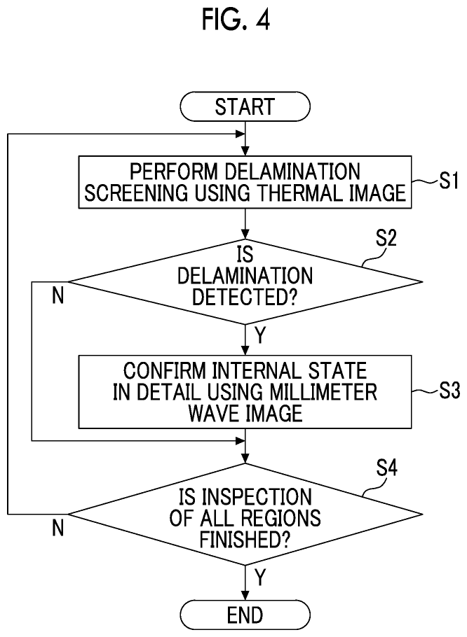

FIG. 4 is a flowchart showing the inspection procedure of the present embodiment.

First, delamination screening using the thermal image is performed (step S1). In this processing, first, the infrared camera 10 is used to capture the thermal image of the surface of the structure O as the inspection target. In the inspection system of the present embodiment, the visible light image is also captured by the visible light camera 20 at the same time as the thermal image is captured. “The same time” here is a concept including a range that is recognized to be substantially the same. The thermal image and the visible light image, which are captured, are output to the inspection device body 40. The inspection device body 40 takes in the thermal image and the visible light image output from the infrared camera 10 and the visible light camera 20, and displays the images on the display unit 47, in response to an instruction from the user. The thermal image and the visible light image are displayed by, for example, being juxtaposed on the same screen. With this, in a case where delamination is detected on the thermal image, the position thereof can be easily specified. In a case where the processing of automatically detecting delamination has been performed on the thermal image, the result thereof is also displayed. For example, the detected delamination region (the region estimated to have delamination) is displayed by being surrounded by a frame. In this case, the visible light image is also displayed with the corresponding region surrounded by a frame. With this, it is possible to easily specify the delamination region on the visible light image. The user confirms the image displayed on the display unit 47 of the inspection device body 40 and performs delamination screening. That is, the region estimated to have delamination is detected. The region estimated to have delamination is an example of the first region. As described above, a temperature difference occurs between the delamination region and the surrounding sound region thereof. Therefore, it is possible to discriminate the region estimated to have delamination by observing the thermal image.

As a result of screening, the presence or absence of the delamination within an inspection target region is determined (step S2). The detection target region is a region captured by the infrared camera 10.

In a case where the delamination is detected from the thermal image, processing of measuring the internal state is performed. In the present embodiment, the internal state of the target region is confirmed in detail by capturing the millimeter wave image (step S3). This processing is performed in the following procedure. First, the millimeter wave camera 30 images the region detected to have delamination. In this case, a region including the location detected to have delamination is set as a detailed confirmation target region, and the set region is imaged by the millimeter wave camera 30. The detailed confirmation target region is set as a partial region in the thermal image. For example, a region where the region detected to have delamination is surrounded by a rectangular frame is set as the detailed confirmation target region. In a case where the detailed confirmation target region exceeds the imaging range of the millimeter wave camera 30, imaging is performed a plurality of times. That is, the detailed confirmation target region is divided into a plurality of regions, and imaging is performed for each region. The captured millimeter wave image is output to the inspection device body 40. The inspection device body 40 takes in the millimeter wave image output from the millimeter wave camera 30 and displays the millimeter wave image on the display unit 47, in response to an instruction from the user. The millimeter wave image to be displayed is an image that visualizes the internal state of concrete, and it is possible to confirm the presence or absence and the state of the delamination in detail by confirming the image.

After the detailed confirmation, whether or not the inspection of all the regions is finished is determined (step S4). That is, whether or not the inspection of all the regions as the inspection target is completed is determined. In a case where the inspection of all the regions is completed, the inspection processing is finished. On the other hand, in a case where the inspection of all the regions is not completed, the process returns to step S1 and the series of processing is repeatedly executed.

As described above, in the inspection method of the present embodiment, the delamination is detected on the basis of the thermal image, and the internal state is confirmed in detail only in a case where the delamination is detected. With this, a highly accurate inspection can be efficiently performed. That is, although the inspection of delamination using the thermal image can easily inspect a wide range, there is a problem in resolution. In that respect, the region detected to have delamination in the thermal image is inspected using the millimeter wave image so that the internal state thereof can be known in detail. Further, since the capturing of the millimeter wave image is limited to the partial region, the imaging can be finished in a short time. With this, both accuracy and time are compatible with each other, and a highly accurate inspection can be efficiently performed.

### Second Embodiment

In the present embodiment, in a case where the delamination is detected from the thermal image, an inspection through hammering is performed. In a case where no abnormality is detected in the inspection through hammering, work to confirm the internal state in detail is performed by capturing the millimeter wave image. For example, an inspector hits the corresponding location with a hammer for an inspection and confirms the presence or absence of flaking, whereby the inspection through hammering is performed.

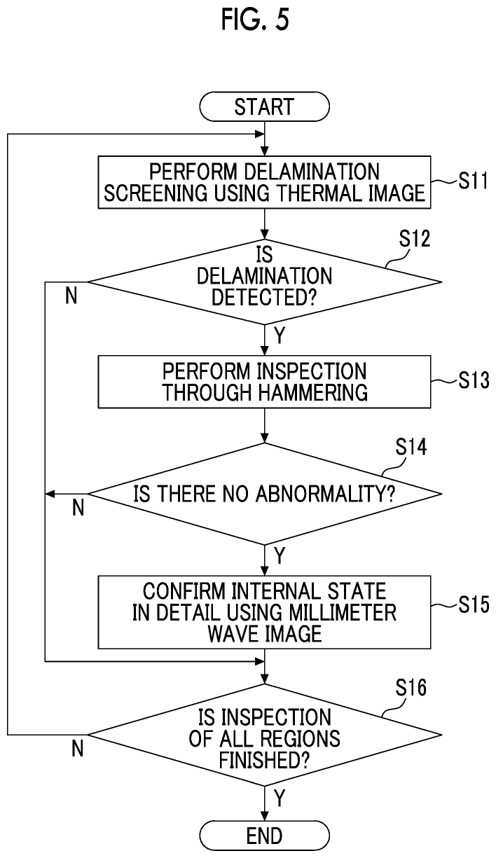

FIG. 5 is a flowchart showing the inspection procedure of the present embodiment.

First, delamination screening using the thermal image is performed (step S11). As a result of screening, the presence or absence of the delamination within the inspection target region is determined (step S12).

In a case where the delamination is detected from the thermal image, the inspection through hammering is performed (step S13). That is, work to confirm the presence or absence of flaking is performed by hitting the region detected to have delamination with the hammer for an inspection.

As a result of the inspection through hammering, the presence or absence of the abnormality is determined (step S14). Here, the presence or absence of flaking is determined. In a case where there is no flaking, detailed confirmation work of the internal state using the millimeter wave image is performed (step S15). That is, the region detected to have delamination is imaged by the millimeter wave camera 30, and work to confirm the internal state in detail is performed using the millimeter wave image obtained by the imaging.

After the detailed confirmation, whether or not the inspection of all the regions is finished is determined (step S16). In a case where the inspection of all the regions is completed, the inspection processing is finished. On the other hand, in a case where the inspection of all the regions is not completed, the process returns to step S11 and the series of processing is repeatedly executed.

As described above, in the inspection method of the present embodiment, the delamination is detected on the basis of the thermal image, and the inspection through hammering is performed in a case where the delamination is detected. In a case where no abnormality is detected by the inspection through hammering, the internal state thereof is confirmed in detail by capturing the millimeter wave image. With this, a highly accurate inspection can be more efficiently performed. That is, in a case where flaking occurs in the inspection through hammering, the subsequent detailed inspection can be omitted because there is a clear abnormality. On the other hand, in a case where no flaking occurs in the inspection through hammering, it is necessary to confirm the internal state in detail. In this case, since the millimeter wave image is captured, the internal state can be confirmed in detail.

In the present embodiment, a configuration is adopted in which the presence or absence of the abnormality is inspected on the basis of the presence or absence of flaking in the hammered location in performing the inspection through hammering, but a configuration can also be adopted in which the presence or absence of the abnormality is inspected through acoustic impact (a so-called acoustic impact test). In this case, in a case where no abnormality is detected by the inspection through acoustic impact, the millimeter wave image is captured.

### Third Embodiment

In the present embodiment, whether or not a detailed inspection using the millimeter wave image is necessary is determined on the basis of both the thermal image and the visible light image. Specifically, delamination is detected in both the thermal image and the visible light image, and a detailed inspection using the millimeter wave image is performed in a case where the delamination is detected from both the images.

The detection of delamination from the visible light image is performed by detecting, for example, a water leakage location. Here, water leakage (including rust juice) is a phenomenon in which moisture, rainwater, and the like in concrete leak out through fissuring, joints, masonry joints, scaling portions, and the like to the outside. Therefore, it is possible to detect the region estimated to have delamination by detecting the water leakage location from the visible light image. The water leakage location is an example of the second region.

It is also possible to add a configuration in which the water leakage location is automatically detected from the visible light image. For example, a technique for automatically detecting the water leakage location from the image using an image recognition model generated through machine learning, deep learning, or the like can be adopted.

In a case where the water leakage location is automatically detected from the visible light image, the processing thereof is performed by the image processing unit 40B. In a case where the water leakage location is automatically detected from the visible light image, the image processing unit 40B generates an image including the detection result, as the image for display. For example, in the visible light image, an image in which the detected water leakage location is surrounded by a frame is generated as the image for display.

The region where water leakage occurs has a lower temperature than the surrounding sound region. Therefore, it is possible to detect the water leakage location also from the thermal image. That is, it is possible to detect a region estimated to have water leakage by detecting a region with a relatively lower temperature than the surrounding region. In the present embodiment, as one of the regions estimated to have delamination, the region with a relatively lower temperature than the surrounding region is detected from the thermal image.

The thermal image and the visible light image are individually displayed on the display unit 47 in response to an instruction from the user. Alternatively, the thermal image and the visible light image are displayed by being juxtaposed on the same screen, in response to an instruction from the user. The user can discriminate the water leakage location by confirming the display on the display unit 47.

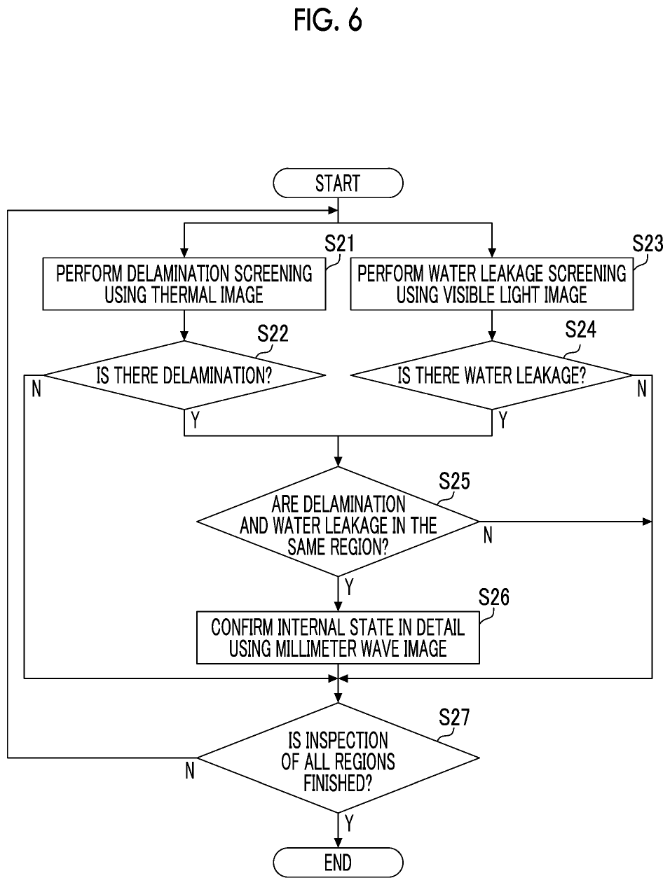

FIG. 6 is a flowchart showing the inspection procedure of the present embodiment.

First, delamination screening using the thermal image is performed (step S21). As a result of screening, the presence or absence of the delamination within the inspection target region is determined (step S22).

In parallel with the delamination screening using the thermal image, water leakage screening using the visible light image is performed (step S23). The water leakage screening using the visible light image is performed in the following procedure. First, the visible light camera 20 is used to capture the visible light image of the surface of the structure O as the inspection target. In the inspection system of the present embodiment, the visible light image is captured at the same time as the thermal image is captured. The captured visible light image is output to the inspection device body 40. The inspection device body 40 takes in the visible light image output from the visible light camera 20 and displays the visible light image on the display unit 47, in response to an instruction from the user. The user confirms the image displayed on the display unit 47 of the inspection device body 40 and performs water leakage screening. As a result of screening, the presence or absence of the water leakage within the inspection target region is determined (step S24).

As described above, the visible light image and the thermal image are displayed by being juxtaposed on the same screen, in response to an instruction from the user. Alternatively, the visible light image and the thermal image are individually displayed in response to an instruction from the user.

In a case where delamination is detected from the thermal image and water leakage is detected from the visible light image, whether or not the delamination and the water leakage are at the same location is determined (step S25). That is, whether or not the delamination region detected in the thermal image and the water leakage region detected from the visible light image are the same region is determined. The same region here includes regions that are recognized to be substantially the same.

In the case of the same region, detailed confirmation work of the internal state using the millimeter wave image is performed (step S26). That is, the region detected to have delamination and water leakage is imaged by the millimeter wave camera 30, and work to confirm the internal state in detail is performed using the millimeter wave image obtained by the imaging.

After the detailed confirmation, whether or not the inspection of all the regions is finished is determined (step S27). In a case where the inspection of all the regions is completed, the inspection processing is finished. On the other hand, in a case where the inspection of all the regions is not completed, the process returns to step S21 and the series of processing is repeatedly executed.

As described above, in the inspection method of the present embodiment, whether or not a detailed inspection using the millimeter wave image is necessary is determined on the basis of both the thermal image and the visible light image. With this, a highly accurate inspection can be efficiently performed.

In the present embodiment, a configuration is adopted in which the region estimated to have delamination is detected by detecting the water leakage location from the visible light image, but the type of abnormality detected from the visible light image is not limited thereto. The type of abnormality need only be an abnormality (damage) of a type in which delamination is estimated to occur. For example, it is possible to detect the region estimated to have delamination by detecting a predetermined pattern of fissuring, free lime, discoloration of concrete, or the like. Free lime is a phenomenon in which a component in concrete, such as calcium oxide, leaks out together with moisture, such as rainwater, to the outside.

Further, for the visible light image, for example, it is also possible to adopt a technique for automatically detecting the abnormal location on the basis of the brightness distribution and/or the RGB value distribution of the image. Since the abnormal location has brightness distribution and RGB value distribution different from other regions, it is possible to automatically detect the abnormal location from the visible light image by searching for changes in brightness values and/or RGB values.

Further, in a case where the abnormal location is automatically detected from the visible light image, it is possible to add processing of discriminating the type of detected abnormality. That is, it is also possible to add processing of discriminating the type of abnormality, such as fissuring, water leakage, and free lime. This processing can be performed using, for example, an image recognition model generated through machine learning, deep learning, or the like.

### Fourth Embodiment

Here, the processing after confirming the internal state in detail using the millimeter wave image will be described.

In the structure made of reinforced concrete, there is a probability of the corrosion of the reinforcing bar being progressing in a case where the internal delamination is large.

In a case where delamination of a predetermined size or more is detected in inspecting the internal state in detail using the millimeter wave image, the soundness of the surrounding reinforcing bar thereof is inspected with emphasis. This can be useful for estimating deterioration factors or for supporting subsequent repair design.

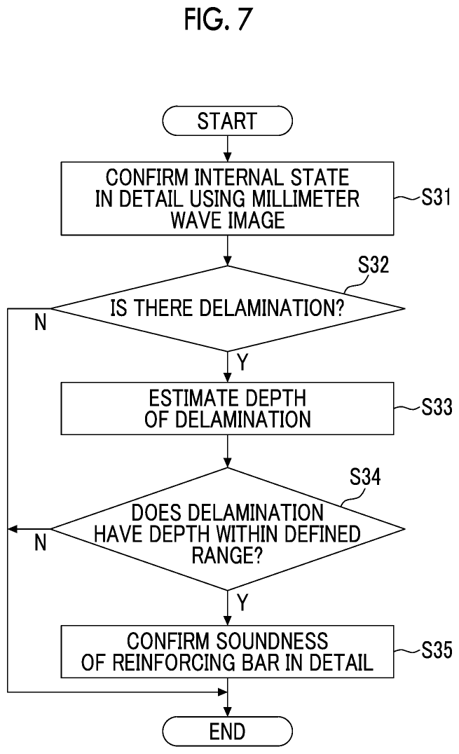

FIG. 7 is a flowchart showing the inspection procedure of the present embodiment.

First, detailed confirmation work of the internal state using the millimeter wave image is performed (step S31). That is, the inspection target region is imaged by the millimeter wave camera 30, and work to confirm the internal state in detail is performed using the millimeter wave image obtained by the imaging. The inspection target region is a region estimated to have delamination. For example, the inspection target region is within a certain range around the center or the centroid of the delamination region. It is possible to detect the presence or absence of the delamination in detail by confirming the internal state in detail using the millimeter wave image.

As a result of the confirmation work, whether or not delamination is detected is determined (step S32). In a case where delamination is detected, the depth of the occurring delamination is estimated from the millimeter wave image (step S33).

Whether or not the estimated depth of delamination is a depth within a defined range is determined (step S34). The defined range is set on the basis of the covering thickness of the concrete of the structure as the inspection target. In a case where the covering thickness of the concrete of the structure as the inspection target is, for example, 4 to 5 cm, the defined range is set to 4 to 5 cm.

In a case where the estimated depth of delamination is within the defined range, work to confirm the soundness of the reinforcing bar in detail is performed (step S35). A non-destructive inspection method is adopted for inspecting the soundness of the reinforcing bar. The inspection of the soundness is performed by, for example, an electromagnetic induction method, or an electromagnetic wave radar method.

The electromagnetic induction method is a method of radiating magnetic lines of force (primary magnetic field) from a magnetic field generation unit of a exploration device toward the concrete, detecting a secondary magnetic field caused by an induced current generated in a conductive substance (reinforcing bar) existing in the concrete through an electromagnetic detection unit, and comparing the increase and decrease of the primary magnetic field and the secondary magnetic field with each other, to detect the reinforcing bar and measure the position thereof.

The electromagnetic wave radar method is a method of receiving an electromagnetic wave reflected at the interface with a substance (reinforcing bar) having different electrical properties through a reception unit in a case where the electromagnetic wave is radiated from a transmission unit of a exploration device toward the concrete, to detect the reinforcing bar.

As described above, in the present embodiment, in a case where delamination is detected in inspecting the internal state in detail using the millimeter wave image, the soundness of the surrounding reinforcing bar thereof is inspected as necessary. With this, the internal state of the abnormal location can be inspected in more detail.

In the present embodiment, the electromagnetic induction method, the electromagnetic wave radar method, and the like are adopted as the method of non-destructively inspecting the soundness of the reinforcing bar, but the method of non-destructively inspecting the soundness of the reinforcing bar is not limited thereto. In addition, for example, a radiation transmission method, an ultrasonic method, or the like can also be adopted.

### OTHER EMBODIMENTS

[Unit that Measures Internal State of Structure]

In the above embodiment, the internal state of the structure is measured by visualizing the internal state of the structure using the millimeter wave camera, but the method of measuring the internal state of the structure is not limited thereto. For example, the internal state of the structure can also be measured using a device (such as a microwave imaging device, a terahertz imaging device, and an ultrasonic imaging device) that visualizes the internal state using an electromagnetic wave, such as a microwave and a terahertz wave, or an ultrasonic wave. Further, the internal state of the structure can also be measured by adopting a non-destructive inspection method, such as a non-contact acoustic exploration method. In addition, a well-known non-destructive exploration method can be adopted.

[Inspection Target]

The present invention is particularly effective in a case where a structure made of reinforced concrete, such as a bridge, a tunnel, a dam, and a building, is inspected, but the application of the present invention is not limited thereto. In addition, the same can also be applied to, for example, a structure whose surface includes a tile, a brick, or the like.

Further, in the above embodiment, a case where the presence or absence of the delamination of concrete is inspected has been described as an example, but the abnormality (damage) as the inspection target is not limited thereto. The present invention is particularly effective for inspecting an internal abnormality that is difficult to visually recognize from the surface.

[Imaging Method]

Imaging with the infrared camera, the visible light camera, and the millimeter wave camera can also be performed by mounting the infrared camera, the visible light camera, and the millimeter wave camera on an unmanned aerial vehicle (a so-called drone), an unmanned traveling vehicle, or the like.

Further, a configuration can also be adopted in which the imaging is automatically performed in a case where the infrared camera, the visible light camera, and the millimeter wave camera are mounted on the unmanned aerial vehicle or the like to image the inspection target. For example, a configuration may be adopted in which the unmanned aerial vehicle automatically flies along a predetermined route and images the inspection target.

[System Configuration]

In the above embodiment, the inspection device body is realized by a so-called stand-alone computer, but the inspection device body can also be realized by a client-server system. For example, the server may have a function of automatically detecting delamination or the like from the thermal image and the visible light image.

Further, the hardware that realizes the inspection device body can be composed of various processors. The various processors include, for example, a CPU and/or a graphic processing unit (GPU) which is a general-purpose processor that executes a program to function as various processing units, a programmable logic device (PLD), such as a field programmable gate array (FPGA), which is a processor having a changeable circuit configuration after manufacture, a dedicated electric circuit, such as an application specific integrated circuit (ASIC), which is a processor having a dedicated circuit configuration designed to execute specific processing. One processing unit constituting an inspection support device may be composed of one of the above various processors or two or more of the above various processors of the same type or different types. For example, one processing unit may be composed of a combination of a plurality of FPGAs or a combination of a CPU and an FPGA. Alternatively, a plurality of processing units may be composed of one processor. A first example of the configuration in which a plurality of processing units are composed of one processor includes an aspect in which one or more CPUs and software are combined to constitute one processor, and the processor functions as the plurality of processing units, as represented by a computer, such as a client or a server. A second example is an aspect in which a processor that realizes the functions of the entire system including a plurality of processing units with one integrated circuit (IC) chip is used, as represented by system on chip (SoC) and the like. As described above, various processing units are composed of one or more of the various processors described above as the hardware structure. Further, more specifically, an electric circuit (circuitry) in which circuit elements, such as semiconductor elements, are combined is used as the hardware structure of these various processors.

### EXPLANATION OF REFERENCES

1: inspection system
10: infrared camera
20: visible light camera
30: millimeter wave camera
40: inspection device body
40A: image acquisition unit
40B: image processing unit
40C: display control unit
41: CPU
42: RAM
43: ROM
44: HDD
45: communication interface
46: operation unit
47: display unit
O: structure
S1 to S4: inspection procedure
S11 to S16: inspection procedure
S21 to S27: inspection procedure
S31 to S35: inspection procedure

What is claimed is:

1. A structure inspection method comprising:
capturing a thermal image of a structure with an infrared camera;
detecting a first region estimated to have an internal abnormality, on the basis of the thermal image; and
measuring an internal state of the first region in a case where the first region is detected.

2. The structure inspection method according to claim 1,
wherein the internal state of the first region is measured by capturing an image that visualizes the internal state of the first region, in a case where the first region is detected.

3. The structure inspection method according to claim 2,
wherein the internal state of the first region is measured by capturing the image that visualizes the internal state of the first region using an electromagnetic wave or an ultrasonic wave, in a case where the first region is detected.

4. The structure inspection method according to claim 3,
wherein the internal state of the first region is measured by capturing the image that visualizes the internal state of the first region using a millimeter wave, a microwave, or a terahertz wave, in a case where the first region is detected.

5. The structure inspection method according to claim 1,
wherein the internal state of the first region is measured by a non-contact acoustic exploration method in a case where the first region is detected.

6. The structure inspection method according to claim 1, further comprising:
measuring the internal state of the first region by hammering the first region, in a case where the first region is detected,
wherein the internal state of the first region is measured in a case where determination is made that there is no abnormality after the first region is hammered.

7. The structure inspection method according to claim 6,
wherein determination is made that there is no abnormality in a case where the first region has no flaking after the first region is hammered.

8. The structure inspection method according to claim 1,
wherein in a case where the structure is a structure made of reinforced concrete,
a region estimated to have delamination is detected as the first region on the basis of the thermal image.

9. The structure inspection method according to claim 1, further comprising:
capturing a visible light image of a surface of the structure with a visible light camera; and
detecting a second region estimated to have an internal abnormality, on the basis of the visible light image,
wherein the internal state of the first region is measured, in a case where the first region and the second region are detected and the first region and the second region are the same region.

10. The structure inspection method according to claim 9,
wherein the internal state of the first region is measured by capturing an image that visualizes the internal state of the first region, in a case where the first region is detected.

11. The structure inspection method according to claim 10,
wherein the internal state of the first region is measured by capturing the image that visualizes the internal state of the first region using an electromagnetic wave or an ultrasonic wave, in a case where the first region is detected.

12. The structure inspection method according to claim 11,
wherein the internal state of the first region is measured by capturing the image that visualizes the internal state of the first region using a millimeter wave, a microwave, or a terahertz wave, in a case where the first region is detected.

13. The structure inspection method according to claim 9,
wherein the internal state of the first region is measured by a non-contact acoustic exploration method in a case where the first region is detected.

14. The structure inspection method according to claim 9,
wherein in a case where the structure is a structure made of reinforced concrete,
a region estimated to have delamination is detected as the first region on the basis of the thermal image, and
a water leakage region is detected as the second region on the basis of the visible light image.

15. The structure inspection method according to claim 14, further comprising:
inspecting soundness of a reinforcing bar in the first region, in a case where the delamination is detected in the first region as a result of measuring the internal state of the first region and a depth of the delamination is within a predetermined range.

16. The structure inspection method according to claim 15,
wherein the soundness of the reinforcing bar is non-destructively inspected.

17. The structure inspection method according to claim 16,
wherein the soundness of the reinforcing bar is inspected by an electromagnetic induction method.

18. A structure inspection method comprising:
measuring an internal state of an inspection target region for a structure made of reinforced concrete; and
inspecting soundness of a reinforcing bar in the inspection target region, in a case where delamination is detected in the inspection target region by the measurement and a depth of the detected delamination is within a predetermined range.

19. The structure inspection method according to claim 18,
wherein the soundness of the reinforcing bar is non-destructively inspected.

20. The structure inspection method according to claim 19,
wherein the soundness of the reinforcing bar is inspected by an electromagnetic induction method.

21. A structure inspection system comprising:
an infrared camera that captures a thermal image of a structure; and
an imaging device that captures an image which visualizes an internal state of the structure,
wherein in a case where a region estimated to have an internal abnormality in the structure is detected from the thermal image captured by the infrared camera, an internal state of the region is measured by capturing an image that visualizes the internal state of the region with the imaging device.
